(19) United States
(12) Patent Application Publication
(10) Pub. No.: US 2024/0374713 A1
(43) Pub. Date: Nov. 14, 2024

(54) SARS-COV-2 IMMUNIZING COMPOSITIONS AND METHODS

(71) Applicant: UNM RAINFOREST INNOVATIONS, Albuquerque, NM (US)

(72) Inventors: David S. Peabody, Albuquerque, NM (US); Bryce Chackerian, Albuquerque, NM (US); Steven Bradfute, Albuquerque, NM (US); Julianne Peabody, Albuquerque, NM (US)

(21) Appl. No.: 18/660,537

(22) Filed: May 10, 2024

Related U.S. Application Data
(60) Provisional application No. 63/465,912, filed on May 12, 2023.

(51) Int. Cl. A61K 39/215; A61K 39/00; A61K 45/06; C12N 7/00

(52) U.S. Cl. CPC A61K 39/215; A61K 45/06; C12N 7/00; A61K 2039/5258; A61K 2039/575; C12N 2770/20034; C12N 2795/00022; C12N 2795/00034

(57) ABSTRACT

In one aspect, an immunogen includes an immunogenic carrier comprising an RNA bacteriophage virus-like particle (VLP) and an antigenic SARS-CoV-2 stem-helix peptide linked to the immunogenic carrier. In another aspect, an immunogen includes an immunogenic carrier comprising an RNA bacteriophage virus-like particle (VLP) and an antigenic SARS-CoV-2 nucleocapsid (N) protein peptide linked to the immunogenic carrier. In one or more embodiments of either aspect, the immunogen may be a component of a pharmaceutical composition. In one or more embodiments of either aspect, the immunogen may be a component of a vaccine.

### CROSS REFERENCE TO RELATED APPLICATIONS

This application claims the benefit of 63/465,912, filed May 12, 2023, the disclosure of which is incorporated by reference herein in its entirety.

### GOVERNMENT FUNDING

This invention was made with government support under CA118110 awarded by the National Institutes of Health. The government has certain rights in the invention.

### SEQUENCE LISTING

This application contains a Sequence Listing electronically submitted via Patent Center to the United States Patent and Trademark Office as an XML file entitled “SequenceListing0310.000182US01” having a size of 15 kilobytes and created on May 6, 2024. The information contained in the Sequence Listing is incorporated by reference herein.

### SUMMARY

This disclosure describes, in one aspect, an immunogen that includes an immunogenic carrier comprising an RNA bacteriophage virus-like particle (VLP) and an antigenic SARS-CoV-2 stem-helix peptide linked to the immunogenic carrier. In one or more embodiments, the RNA VLP is MS2 bacteriophage. In one or more embodiments, the RNA VLP is PP7 bacteriophage.

In one or more embodiments, the SARS-CoV-2 stem-helix peptide includes a segment having at least 75% sequence similarity to an antigenic portion of SEQ ID NO:1.

In another aspect, this disclosure describes a composition that includes an immunogen that includes an immunogenic carrier comprising an RNA bacteriophage virus-like particle (VLP) and an antigenic SARS-CoV-2 stem-helix peptide linked to the immunogenic carrier. In one or more embodiments, the composition further includes an adjuvant.

In another aspect, this disclosure describes an immunogen that includes an immunogenic carrier comprising an RNA bacteriophage virus-like particle (VLP) and an antigenic SARS-CoV-2 nucleocapsid (N) protein peptide linked to the immunogenic carrier. In one or more embodiments, the RNA VLP is MS2 bacteriophage. In one or more embodiments, the RNA VLP is PP7 bacteriophage.

In one or more embodiments, the antigenic SARS-CoV-2 N-protein peptide comprises a segment having at least 75% sequence similarity to any one of SEQ ID NOs:6-14.

In another aspect, this disclosure describes a composition that includes an immunogen that includes an immunogenic carrier comprising an RNA bacteriophage virus-like particle (VLP) and an antigenic SARS-CoV-2 nucleocapsid (N) protein peptide linked to the immunogenic carrier. In one or more embodiments, the composition further includes an adjuvant.

In another aspect, this disclosure describes a composition that includes a first population of VLPs displaying an antigenic SARS-CoV-2 stem-helix peptide and a second population of VLPs displaying an antigenic SARS-CoV-2 peptide that differs from the SARS-CoV-2 stem-helix peptide displayed by the first population of VLPs. In one or more embodiments, the composition further includes an adjuvant.

In another aspect, this disclosure describes a method of increasing serum titer of anti-SARS-CoV-2 antibodies in a subject. Generally, the method includes administering to the subject a therapeutically effective amount of a composition that includes an immunogenic carrier comprising an RNA bacteriophage virus-like particle (VLP) and an antigenic SARS-CoV-2 stem-helix peptide linked to the immunogenic carrier.

In one or more embodiments, the composition further includes a second population of immunogenic carriers comprising an RNA bacteriophage virus-like particle (VLP) and an antigenic SARS-CoV-2 peptide that differs from the SARS-CoV-2 stem-helix peptide.

In one or more embodiments, the method further includes administering to the individual at least one additional therapeutic agent for treating infection by SARS-CoV-2.

In one or more embodiments, the composition is administered to the individual before the individual manifests a symptom or clinical sign of infection by SARS-CoV-2. In one or more embodiments, the composition is administered to the individual after the individual manifests a symptom or clinical sign of infection by SARS-CoV-2.

In another aspect, this disclosure describes a method of increasing serum titer of anti-SARS-CoV-2 antibodies in a subject. Generally, the method includes administering to the subject a therapeutically effective amount of a composition that includes an immunogenic carrier comprising an RNA bacteriophage virus-like particle (VLP) and an antigenic SARS-CoV-2 nucleocapsid (N) protein peptide linked to the immunogenic carrier.

In one or more embodiments, the composition further includes a second population of immunogenic carriers comprising an RNA bacteriophage virus-like particle (VLP) and an antigenic SARS-CoV-2 peptide that differs from the SARS-CoV-2 N-protein peptide.

In one or more embodiments, the method further includes administering to the individual at least one additional therapeutic agent for treating infection by SARS-CoV-2.

In one or more embodiments, the composition is administered to the individual before the individual manifests a symptom or clinical sign of infection by SARS-CoV-2. In one or more embodiments, the composition is administered to the individual after the individual manifests a symptom or clinical sign of infection by SARS-CoV-2.

In another aspect, this disclosure describes a vaccine that includes an immunogen that includes an immunogenic carrier comprising an RNA bacteriophage virus-like particle (VLP) and an antigenic SARS-CoV-2 stem-helix peptide linked to the immunogenic carrier.

In another aspect, this disclosure describes a vaccine that includes an immunogen that includes an immunogenic carrier comprising an RNA bacteriophage virus-like particle (VLP) and an antigenic SARS-CoV-2 nucleocapsid (N) protein peptide linked to the immunogenic carrier.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Structure of SARS-CoV-2, showing locations of spike proteins and nucleocapsid proteins.

FIG. 2. Structure of SARS-CoV-2 stem-helix and its location in the prefusion form of spike protein.

FIG. 3. Plasmids encoding the N-terminal MS2 and C-terminal PP7 stem-helix fusions. Expression in E. coli produces the genetically modified coat proteins, which dimerize and then assemble into VLPs with multiple copies of the stem-helix trimer displayed on their surfaces.

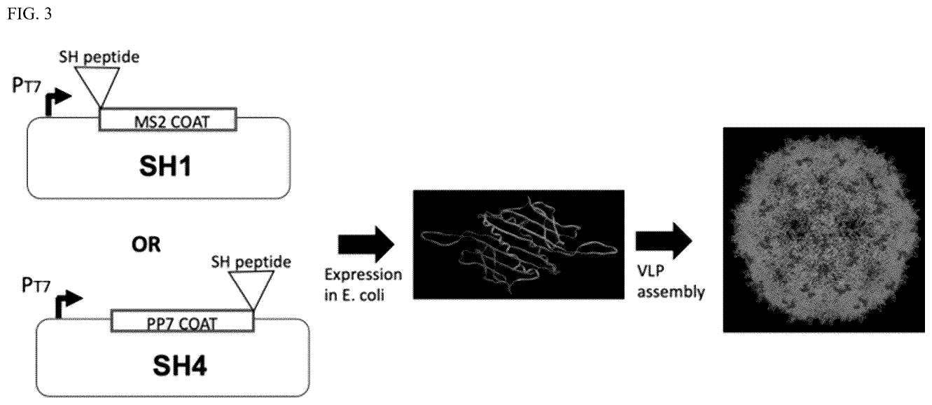

FIG. 4. ELISA showing the presence of high titer anti-spike antibodies in the sera of mice immunized with a mixture of SH1 and SH4 VLPs.

FIG. 5. Hamster challenge study design.

FIG. 6. End-point dilution titers of anti-spike antibodies and anti-nucleocapsid antibodies in hamster sera

FIG. 7. Weight loss after infection.

FIG. 8. Viral load in lungs three days post infection.

FIG. 9. Insertion of foreign peptide into the AB-loop of the MS2 coat protein single chain dimer results in display of the foreign peptide on the VLP surface.

FIG. 10. Construction of antigen fragment libraries on VLPs. Using synthetic oligonucleotides from microchip-based DNA synthesis, each possible 8-mer-to-14-mer peptide from the SARS-CoV-2 structural proteins was inserted into a separate MS2 coat protein AB-loop.

FIG. 11. The process of epitope identification by biopanning MS2 VLP antigen fragment libraries on antibodies from covid19 patient sera.

FIG. 12. Nucleocapsid protein peptide epitopes from MS2 VLP antigen fragment library recognized by antibodies present in sera from multiple SARS-COV-2-infected patients.

FIG. 13. Schematic structure of nucleocapsid protein and the positions of the peptide epitopes. The stably folded RNA binding domain and dimerization domain are flanked by intrinsically disordered domains, the N-terminal domain (NTD), linker domain (LINK), and C-terminal domain (CTD). The positions of epitopes identified by affinity selection on patient sera are indicated by brackets. The numbers above each bracket indicate the boundaries of the VLP-displayed peptides. Epitope 167.176, denoted with an asterisk (*), is a mapped epitope not actually used in a VLP.

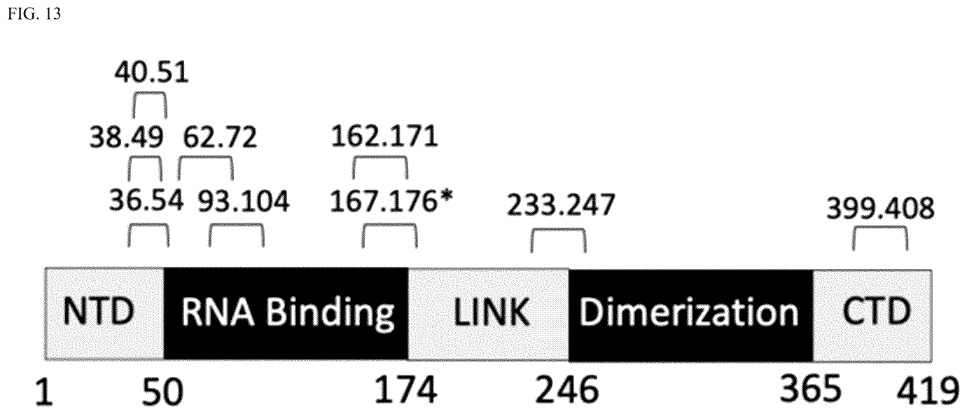

FIG. 14. Immunization of mice with a mixture of VLPs mp26 (38.54), mp30 (93.104), and mp31 (233.245) yields antibodies that bind SARS-CoV-2 nucleoprotein.

FIG. 15. Anti-nucleocapsid ELISA titers in immunized hamster sera.

FIG. 16. Method for insertion of peptides into the AB-loop of the single-chain MS2 coat protein dimer in plasmid pDSP62.

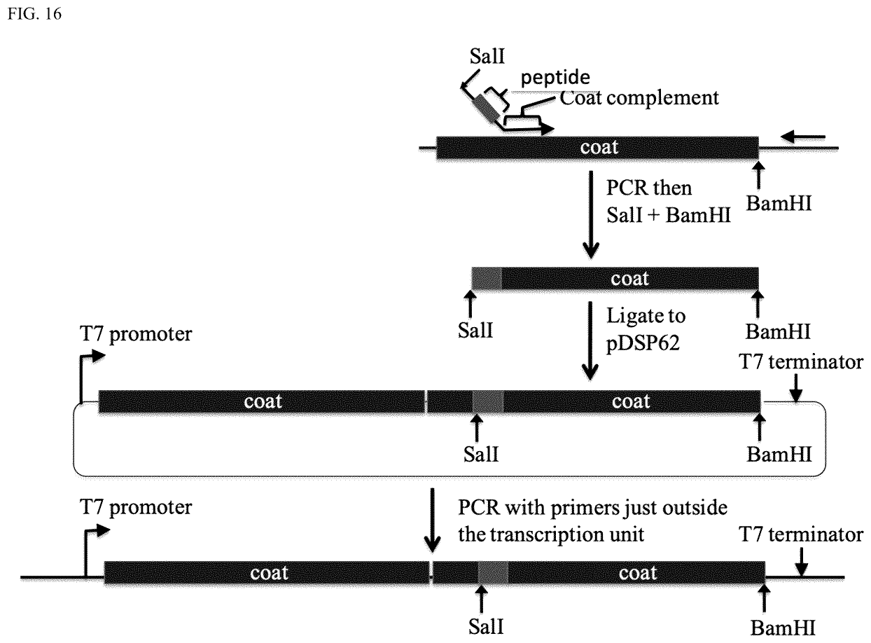

### DETAILED DESCRIPTION OF ILLUSTRATIVE EMBODIMENTS

This disclosure describes new COVID-19 vaccines based on display of one or more SARS-CoV-2 peptides on an RNA bacteriophage virus-like particle (VLP). In one or more embodiments, the SARS-CoV-2 peptide is an antigenic portion of the stem-helix portion of the spike protein. In one or more embodiments, the SARS-CoV-2 peptide is an immunological portion of the nucleocapsid protein.

Most vaccines transiently induce a primary antibody response that typically wanes over the course of weeks or months. When post-vaccination antibody titers decrease to a low residual level, long-term vaccine effectiveness relies on the ability of immune memory to react to subsequent infection with a more rapid secondary response. This delay, however brief, explains why existing COVID-19 vaccines, for example, are better at reducing disease severity than at preventing infection entirely. Inhibiting infection requires the presence of high-titer antibodies at the time of virus exposure. The VLP platform employed in the vaccines described herein elicits high-titer, long-lasting antibody responses.

The stem-helix portion of SARS-CoV-2 spike protein is a known recognition site of broadly neutralizing antibodies but is too poorly immunogenic in its natural context to elicit much of an immune response during normal virus infection. This disclosure describes compositions and methods that artificially enhance the immunogenicity of a stem-helix peptide to make a vaccine that elicits a broadly neutralizing antibody response. A variety of designs are possible but, in the embodiments described here, a 20-amino acid stem-helix peptide is genetically fused at the N-terminus of MS2 coat protein and at the C-terminus of PP7 coat protein. In their respective VLPs, the foreign peptides are brought together at the icosahedral VLP's three-fold symmetry axes where they are encouraged to associate into the same triple helical structure, they normally make in the spike protein trimer. The multiplicity of their display on VLPs renders them sufficiently immunogenic to elicit a high-titer antibody response to spike protein.

The fundamental function of the nucleocapsid (N) protein is to package the viral genome RNA into a long helical ribonucleocapsid (RNP) complex and to participate in the assembly of the virion through its interactions with the viral genome and membrane protein (M). In addition, the N protein of the coronaviruses is involved in the host cellular machinery such as interferon inhibition. RNA interference, and apoptosis, serving a regulatory role in viral life cycles. Moreover, the N protein is also an immunodominant antigen in host immune responses that can be used as a diagnostic antigen and immunogen. Using affinity-selection by human antibodies from a library of antigen fragments on MS2 VLPs, antigenic N-protein peptides were identified that are normally recognized during the immune response to SARS-CoV-2 infection. This disclosure describes construction and synthesis of VLPs displaying the affinity-selected N protein peptides. This disclosure further describes evaluation of the synthetic VLPs, their ability to elicit anti-N-protein antibodies and to treat animals against the effects of SARS-CoV-2 infection.

Current Covid-19 vaccines are effective and safe, but they have shortcomings. First, certain aspects of efficacy are relatively short-lived. After a post-vaccination peak, neutralizing antibodies soon fall to levels below those required for efficacy against infection, explaining why so-called “breakthrough” infections have occurred. Although memory responses offer long-term efficacy against Covid's worst consequences (e.g., hospitalization and death), ideally a vaccine provides efficacy against initial infection. Second, vaccine-induced antibodies can gradually lose efficacy as the virus evolves to escape neutralization. A vaccine that elicits a longer-lasting high-titer antibody response and/or targeted epitopes conserved across a wide spectrum of related viruses can overcome one or both deficiencies.

This disclosure describes vaccines directed against peptides of the stem-helix portion of the SARS-CoV-2 spike protein and/or peptides of the nucleocapsid (N) protein. As used herein, the term vaccine is used to describe an immunizing composition that elicits a clinically relevant immune response—i.e., an immune response that is capable of treating, either prophylactically or therapeutically as described in more detail below, a subject infected or at risk of being infected by an infectious agent.

### Immunizing Compositions Targeting the Stem-Helix of SARS-CoV-2 Spike Protein

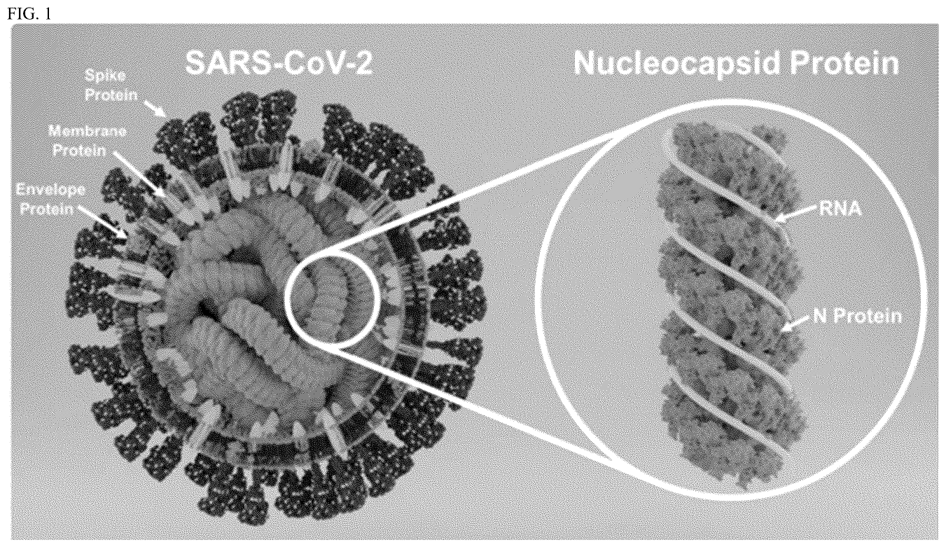

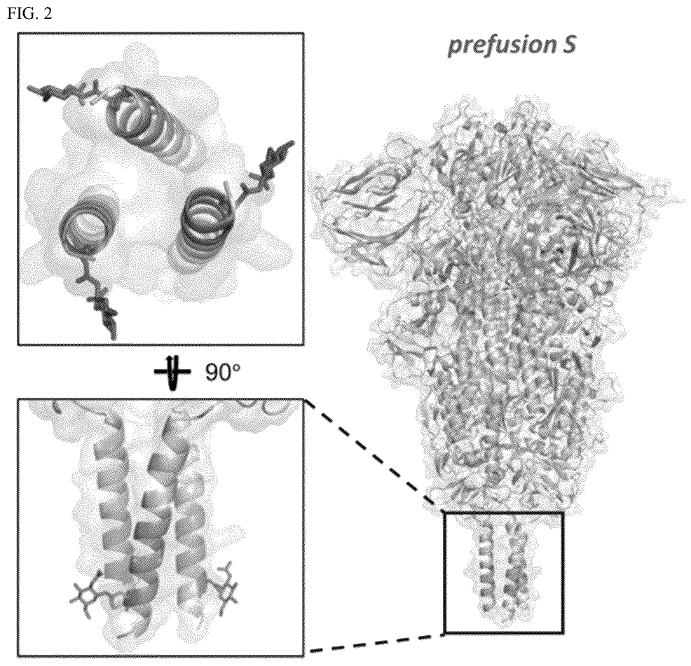

Broadly neutralizing monoclonal antibodies identify the spike protein stem-helix as a potential vaccine target. SARS-CoV-2 neutralizing antibodies, whether produced by vaccination or by natural infection, are normally directed mainly to the spike protein's receptor binding domain (RBD). Genetic mutation of the RBD's immune-dominant epitopes, however, allows emergence of viral variants that escape antibody neutralization. Although antibodies with activity against diverse variants do occasionally occur, the conserved epitopes they recognize are too poorly immunogenic to elicit much of a response. A common target of these broadly neutralizing antibodies is the so-called stem-helix, a 20-amino acid triple helical structure at the base of the trimeric prefusion spike protein (FIG. 1, FIG. 2). Antibody binding to this site interferes with infection, apparently by inhibiting spike-mediated virus-cell fusion. The stem-helix sequence is conserved across SARS-CoV-2 variants and even between a range of different coronaviruses. This disclosure describes the design and construction of an immunizing composition directed against the spike protein stem-helix by artificially increasing the immunogenicity of this normally cryptic site.

### Displaying Stem-Helix Peptides on RNA Bacteriophage Virus-Like Particles (VLPs)

When bacteriophage MS2 coat protein is expressed from a plasmid in E. coli, it self-assembles into an icosahedral particle about 28 nm in diameter. Foreign peptide sequences genetically fused to coat protein end up on the VLP surface where they are displayed in highly immunogenic dense repetitive arrays. This disclosure describes an approach to displaying the SARS-CoV-2 spike protein stem-helix on VLPs.

MS2 and PP7 coat proteins have highly similar three-dimensional structures. Each coat protein folds as a homodimer of two intimately intertwined subunits. Dimers self-assemble to produce the icosahedral VLP in which the N-termini of three different coat proteins are brought into proximity at each of the icosahedron's three-fold symmetry axes. Since the C-termini are close to the N-termini, both ends are similarly arranged around the viral three-fold symmetry axes. The VLPs described herein involve fusing a stem-helix peptide (e.g., SEQ ID NO:1) to either terminus and exploiting the coat protein's natural propensity for trimerization. Two exemplary constructs were synthesized. One fuses the 20-amino acid stem-helix peptide of SEQ ID NO:1 to the MS2 coat protein N-terminus. The second fuses the 20-amino acid stem-helix peptide of SEQ ID NO:1 to the C-terminus of PP7 coat protein. The two different VLP constructs present the stem-helix peptide in different orientations, with either its N-terminus (MS2) or its C-terminus (PP7) pointed away from the particle.

While described herein in the context of exemplary embodiments using MS2 and PP7 VLPs, the constructs and methods described herein can involve using any suitable VLP platform. The virus-like particle (VLP) can include any particle that includes viral proteins assembled to structurally resemble the virus from which they are derived but lack enough of the viral genome so that they are non-replicative and, therefore, noninfectious. A VLP may, therefore, include at least some of the viral genome, but the viral genome is genetically modified so that the viral genes responsible for infectivity and replication are inactivated. Exemplary VLPs include, but are not limited to, VLPs of QP, MS2, PP7, AP205, or other bacteriophage coat proteins, the capsid and core proteins of Hepatitis B virus, measles virus, Sindbis virus, rotavirus, foot-and-mouth-disease virus, Norwalk virus, the retroviral GAG protein, the retrotransposon Ty protein pl, the surface protein of Hepatitis B virus, human papilloma virus, human polyoma virus, RNA phages, Ty, fr-phage, GA-phage, AP 205-phage and, in particular, Qp-phage, Cowpea chlorotic mottle virus, cowpea mosaic virus, human papilloma viruses (HPV), bovine papilloma viruses, porcine parvovirus, parvoviruses such as B19, porcine (PPV) and canine (CPV) parvovirues, caliciviruses (e.g. Norwalk virus, rabbit hemorrhagic disease virus [RHDV]), animal hepadnavirus core Antigen VLPs, filamentous/rod-shaped plant viruses, including but not limited to Tobacco Mosaic Virus (TMV), Potato Virus X (PVX), Papaya Mosaic Virus (PapMV), Alfalfa Mosaic Virus (AIMV), and Johnson Grass Mosaic Virus (JGMV), insect viruses such as flock house virus (FHV) and tetraviruses, polyomaviruses such as Murine Polyomavirus (MPyV), Murine Pneumotropic Virus (MPtV), BK virus (BKV), and JC virus (JCV).

### Construction of the Stem-Helix VLPs

The SARS-CoV-2 stem-helix peptide LQPELDSFKEELDKYFKNHT (SEQ ID NO:1) was reverse translated (with E. coli codon preferences) and then incorporated into PCR primers suitable for fusion to the MS2 coat protein N-terminus or the PP7 coat protein C-terminus. When paired with appropriate primers annealing to the other ends of the MS2 and PP7 sequences, PCR generated coding sequences for the two coat protein/stem-helix fusions SEQ ID NO:2 and SEQ ID NO:3. The PCR products, flanked with NcoI and BamHI sites, were then cloned in plasmid pDSP62 (Chackerian et al., 2011, J Mol. Biol. 409:225-237) under T7 promoter control and expressed in E. coli. SEQ ID NO:2, sometimes referred to herein as the SH1 construct, has the 20-amino-acid stem-helix peptide fused to MS2 coat protein N-terminus through a six-amino-acid linker sequence. SEQ DI NO:3, sometimes referred to herein as the SH4 construct, has the 20-amino-acid stem-helix peptide fused to the PP7 coat protein C-terminus through an eight-amino-acid linker.

### Bacterial Production of the Stem-Helix VLPs

The DNA sequence encoding the SH1 construct (SEQ ID NO:4) and the DNA sequence encoding the SH4 construct (SEQ ID NO:5) are flanked by NcoI and BamHI sites, which makes them easy to insert in place of the MS2 coat protein single-chain dimer sequences in pDSP62 (Chackerian et al., 2011, J. Mol. Biol. 409:225-237), putting them under control of the T7 promoter. The plasmids were introduced into E. coli strain C41(DE3), which were then grown in auto-inducing medium overnight at 30° C. The VLPs were extracted from cells and purified by methods previously described (Peabody et al., 2008, J. Mol. Biol. 380:252-263).

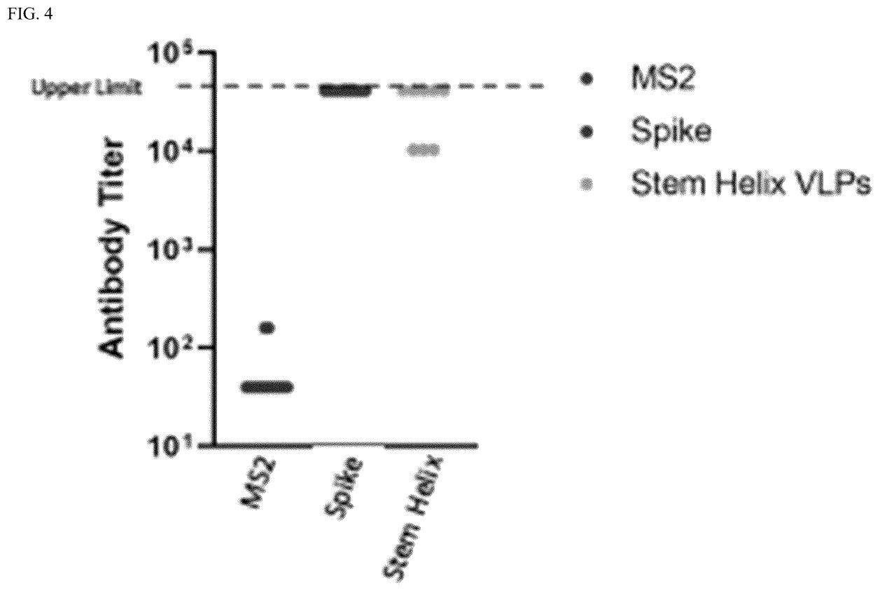

In a preliminary immunogenicity test, C57Bl/6 mice were immunized intramuscularly twice with 10 μg of purified VLPs at three-week intervals. Two weeks after the second immunization anti-spike antibody titers were determined by ELISA. The results are summarized in FIG. 4, which shows end-point dilution titers typically exceeding 10,000.

### Effects of Vaccination on Infection of Hamsters by SARS-CoV-2

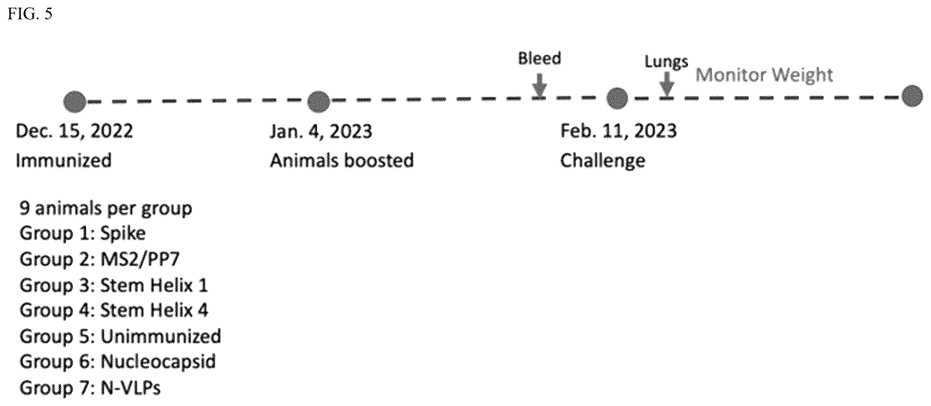

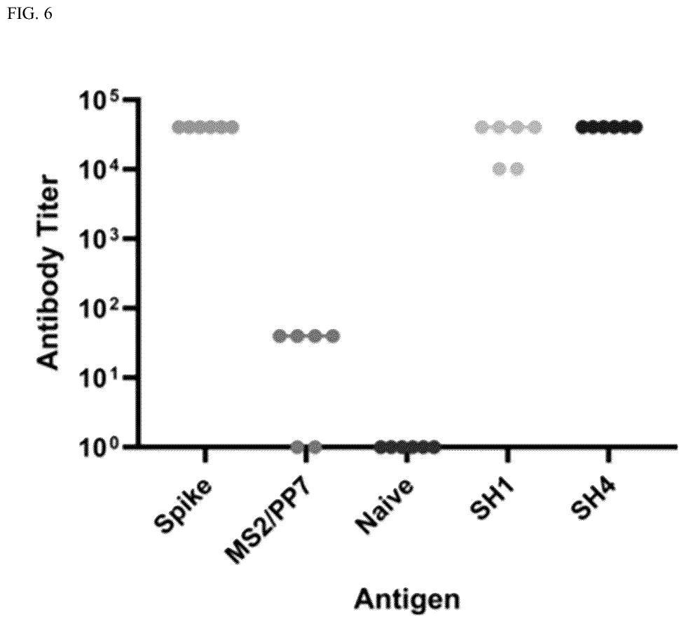

The effects of SH1 and SH4 VLPs on SARS-CoV-2 infection were tested using a hamster model. The schedule of immunization and virus challenge are shown in FIG. 5. A month after the second vaccine dose, blood was drawn, and the sera tested by ELISA for the presence of anti-spike protein antibodies. FIG. 6 shows the end-point dilution titers. A high titer anti-spike response was obtained in each of six animals. Animals in the positive control group were immunized with purified spike protein. Animals in the negative control group were either untreated (naïve) or immunized with a combination of MS2 and PP7 VLPs (no foreign epitope, MS2/PP7).

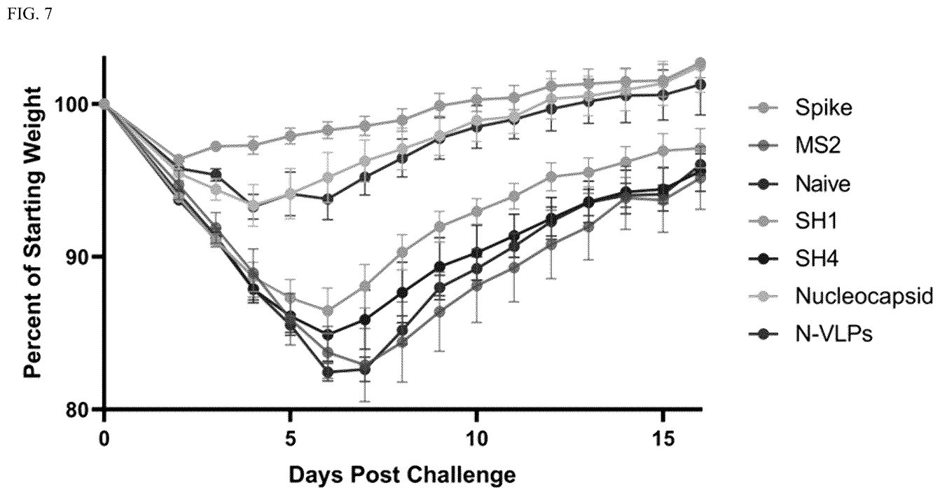

Although SARS-CoV-2 infects hamsters, it doesn't generally kill them. Instead, they lose weight for a few days and then gradually recover. The depth and duration of weight loss and the time to recovery are indications of infection severity. Animals were infected intra-nasally with SARS-CoV-2 and followed the course of infection by daily monitoring of individual animal weights (FIG. 7). Animals immunized with intact spike protein briefly lost a little weight but by day 3 they begin to recover. Naïve animals and animals immunized with the MS2/PP7 VLP (no foreign peptide) lost nearly 20% of their body weights by day 6 and then began their recovery. Vaccination with SH4 and, to a greater extent, SH1 reduced weight loss.

### Alternative Designs

In one or more embodiments, the stem-helix peptide may include one or more amino acid deletions, additions, substitutions, or a combination of such modifications to the amino acid sequence of SEQ ID NO:1. Alternative designs may better mimic the form of the stem-helix peptide that naturally elicits antibodies. Broadly neutralizing monoclonal antibodies that recognize the stem-helix do not typically bind to the intact triple helix. Instead, they typically interact with a single copy of the helical peptide on a surface normally buried in the hydrophobic interior of the triple helix. In other words, it seems the triple helix must dissociate for antibody binding to occur.

Thus, for example, in one or more embodiments, amino acid deletions or substitutions compared to the amino acid sequence of SEQ ID NO:1 may partially destabilize the triple helix, better revealing the interior of the stem-helix that elicits neutralizing antibodies. As another example, in one or more embodiments, the stem-helix peptide may be presented in non-trimeric modes. In one or more of these embodiments, fusing the stem-helix peptide to a single-chain dimer version of coat protein would reduce the overall display density by one-half and create the possibility for display of anywhere from zero to three copies of the stem-helix peptide at each of the sixty three-fold axes. As another example, in one or more embodiments, the stem-helix peptide may be displayed as a lone peptide by fusion to sites further from the three-folds of the coat protein, for example at one of the termini of AP205 coat protein (Peabody et al., 2021, Pharm 14:764). As yet another example, in one or more embodiments, affinity selection by the various broadly neutralizing stem-helix-binding antibodies, either alone or in combinations, using either random sequence or stem-helix mutational libraries, may identify variants to the stem-helix peptide that the antibodies prefer. The stem-helix variants selected in this manner may be better suited to eliciting neutralizing antibodies.

Regardless of the specific design, a vaccine directed against the SARS-CoV-2 stem-helix can provoke new immunity or strengthen and/or prolong a pre-existing immune response (e.g., from prior infection or vaccination) against SARS-CoV-2 by targeting a vulnerable but cryptic virus epitope.

### Immunizing Compositions Targeting Nucleocapsid (N) Protein

The outer surface of SARS-CoV-2 is decorated with the spike protein that mediates cell binding and virus entry. Existing Covid-19 vaccines mimic one aspect of natural infection; they elicit antibodies that bind spike and block entry. But natural infection also elicits antibodies against nucleocapsid protein. This disclosure describes nucleocapsid-targeted alternatives (or perhaps adjuncts) to existing SARS-CoV-2 vaccines that reduces the effects of SARS-CoV-2 infection.

N-protein is not strictly confined to the virus particle or to the interior of infected cells but is also abundant on cell surfaces. This exposure presumably accounts for its immunogenicity and probably also provides the target for anti-N-mediated immunity. Without wishing to be bound by any particular theory or mechanism of action, antibody-dependent cell-mediated cytotoxicity (ADCC) may account for at least some of the effects of antibodies targeting N protein. Alternatively or additionally, some effects may be afforded by the ability of anti-N antibodies to neutralize N-protein-mediated suppression of innate immunity, by inhibiting, for example, its ability to sequester cytokines. Alternatively or additionally, antibody-bound N-protein may interact with the Fc receptor of TRIM21, targeting N-protein for proteolysis that in turn promotes antigen cross-presentation and accelerates a T cell response to infection. Regardless of the specific mechanism or mechanisms employed, anti-nucleocapsid antibodies are more likely to act by limiting an infection's severity than by interfering with virus entry into susceptible cells.

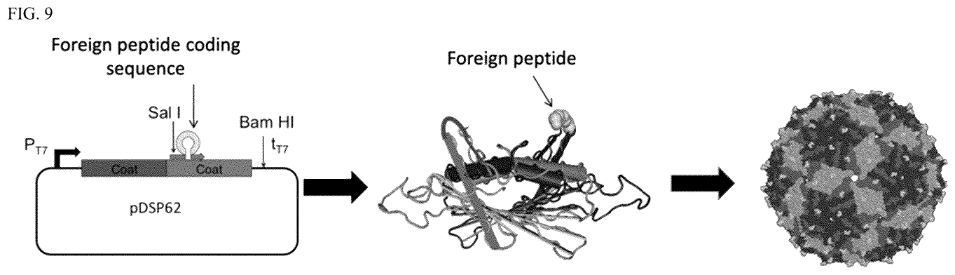

The approach described herein involves display of specific N-protein peptides on RNA bacteriophage virus-like particles. VLPs are produced by self-assembly of plasmid-expressed coat protein into an icosahedral particle about 28 nm in diameter (FIG. 9). When foreign peptide sequences are genetically fused to coat protein, they end up on the VLP surface where they are displayed in a highly immunogenic multivalent format. As vaccines, VLPs typically yield high-titer, long-lived antibody responses to virtually any peptide epitope they display. In contrast, vaccines based on administering purified N protein or an N-encoding mRNA would elicit relatively short-lived antibody responses compared to display of the N-protein on a VLP. Since any vaccine functioning by the mechanisms described above would likely depend on the abundance of antibodies present at the time of infection, durability of the antibody response is beneficial.

### N-Protein Epitopes and Making the VLPs

The MS2 VLP allows both the identification of antibody epitopes using a bio-panning process akin to phage display, and the highly immunogenic display of epitopes as a vaccine. These capabilities depend on two features of the MS2 platform. First, genetic insertion of peptide-encoding sequences into MS2 coat protein's AB-loop results in display of the peptides on the VLP surface (FIG. 9). VLPs are highly immunogenic and when used as a vaccine they efficiently elicit antibodies to the peptides they display. Second, since each MS2 VLP naturally encapsidates its own mRNA, it contains a copy of the genetic information for its own synthesis. After bio-panning, affinity-selected sequences are recovered by reverse transcription and polymerase chain reaction. Sequence analysis determines the identity of affinity-selected peptides.

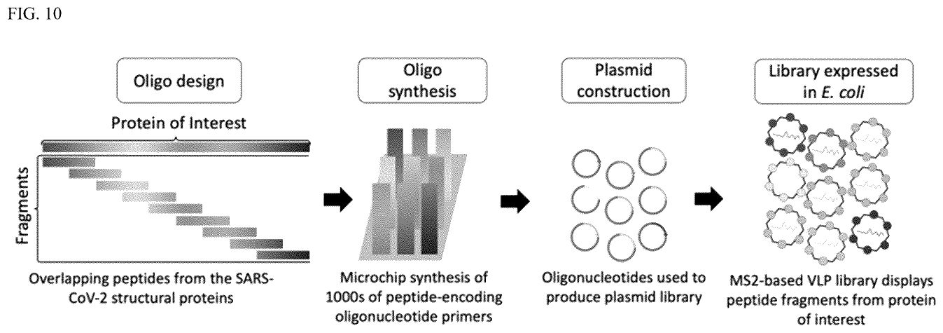

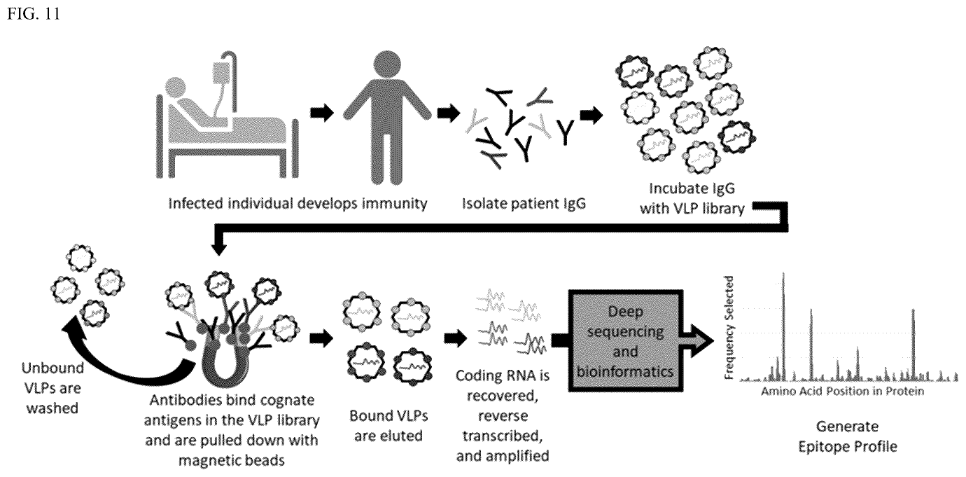

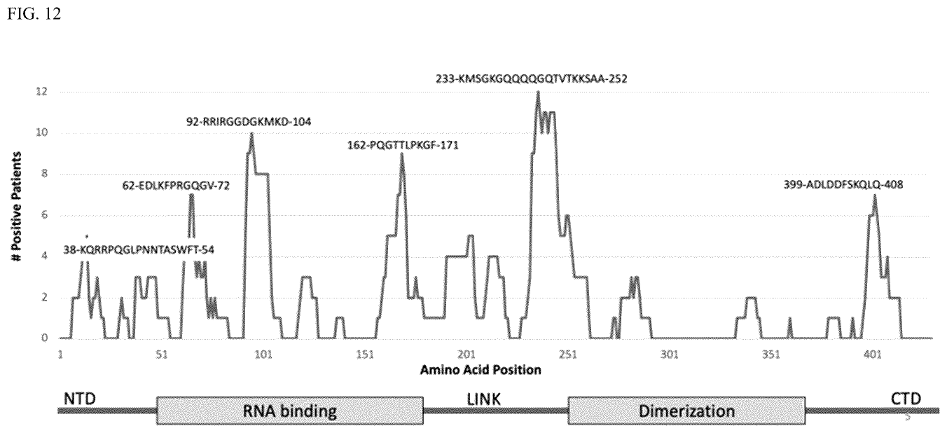

The general scheme of antigen fragment library construction is shown in FIG. 10. Knowledge of the SARS-CoV-2 genome sequence allows one to computationally fragment viral structural proteins into overlapping peptides. Using microchip-based DNA synthesis, one can make oligonucleotides that encode every peptide and insert them into the MS2 coat protein gene. In this case, the antigen-fragment peptide library was constructed by inserting 8-14 amino acid-long peptides that, in aggregate, represent the complete sequences of all four SARS-CoV-2 structural proteins. Expression from plasmids in E. coli produces a library consisting of millions of VLPs displaying many thousands of different antigen fragments. The library was subjected to bio-panning on antibodies from the sera of nineteen SARS-CoV-2-infected humans (FIG. 11). Coding sequences for the selected VLPs were recovered by reverse-transcription and polymerase chain reaction of the RNA contained in the VLPs. The coding sequences were subjected to high-throughput DNA sequence analysis. This yielded many thousands of individual peptide sequences. The degree of enrichment of every peptide in the library was obtained by determining the relative abundance of each peptide before and after affinity-selection. Thus, a profile was developed showing the degree to which each peptide was recognized by each of the nineteen sera. To focus on the most commonly recognized peptides, the number of selections (out of nineteen) were counted in which any given peptide was enriched at least 50-fold. That number was plotted against a peptide's position in the linear N-protein sequence (FIG. 12). Peaks in the plot represent peptides within N-protein recognized by the human antibodies.

Prominent peptides identified are shown in Table 1. Since each is generally defined by a series of overlapping peptide selectants (reflected in peak width in FIG. 12), no one peptide sequence listed in Table I necessarily corresponds perfectly to any individual selectant. For example, the VLP identified as call mp26 displays a 17-amino acid peptide. This is longer than the maximum size (14 amino acids) of any peptide in the antigen-fragment library. The mp26 epitope (38.54) was designed from overlapping selectants to represent the more extensive sequence shown in Table 1.

TABLE 1
Peptide and VLP names with sequences and peptide boundaries

| Peptide | VLP | | | SEQ |
|---|---|---|---|---|
| name | name | AA bounds | Peptide Sequence | ID NO: |
| NP1 | MP26 | 38.54 | 38-KQRRPQGLPNNTASWFT-54 | 6 |
| NP2 | not made | 167.176 | 167-LPKGFYAEGS-176 | 7 |
| NP3 | MP30 | 93.104 | 93-RIRGGDGKMKDL-104 | 8 |
| NP4 | MP31 | 233.245 | 233-KMSGKGQQQQGQT-245 | 9 |
| NP5 | MP27 | 38.49 | 38-KQRRPQGLPNNT-49 | 10 |
| NP6 | MP28 | 40.51 | 40-RRPQGLPNNTAS-51 | 11 |
| Nuc1 | Nuc1 | 62.72 | 62-EDLKFPRGQGV-72 | 12 |
| Nuc2 | Nuc2 | 162.171 | 162-PQGTTLPKGF-171 | 13 |
| Nuc3 | Nuc3 | 399.408 | 399-ADLDDFSKQLQ-408 | 14 |

The nucleocapsid protein is frequently phosphorylated at specific serine and threonine residues, some of which reside in at least two of the peptides of Table 1. Specifically, thr49, ser51, and thr54 of the 38.54 peptide and ser176 of the 167.176 peptide are phosphorylated at least some of the time. Having been produced in E. coli, the VLP-displayed versions of these peptides are devoid of these post-translational modifications. The effect, if any, such modifications might have on the immunogenicity and antibody recognition of these peptides is unknown. Fortunately, advances in genetic code expansion in E. coli make it possible to incorporate phospho-serine and phospho-threonine at these sites if necessary.

### Immunogenicity Testing

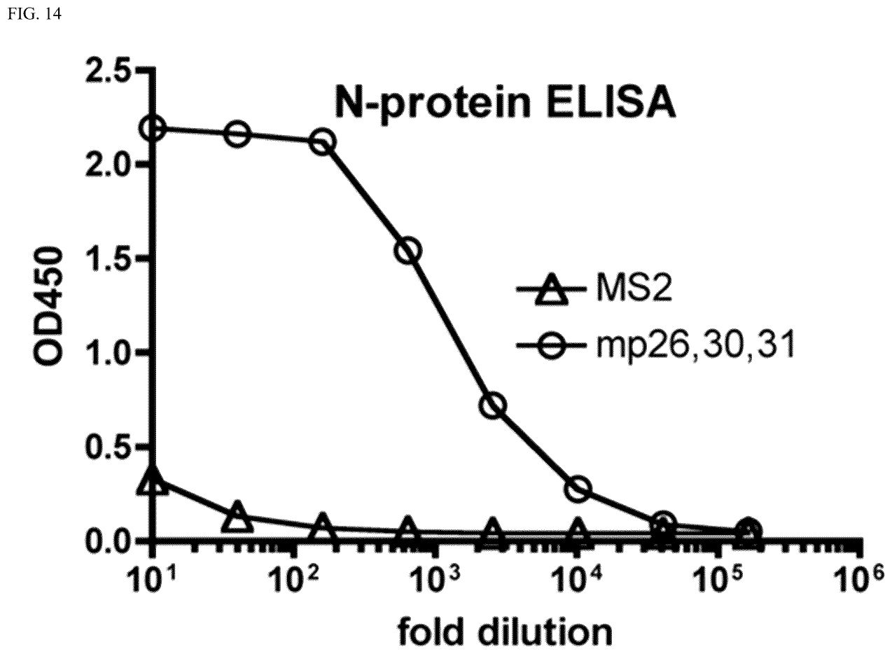

To determine whether selected VLPs could elicit an anti-N antibody response, the mp26, mp30 and mp31 VLPs were produced in bacteria, purified individually, and then mixed together for intramuscular immunization of C57/B6 mice. ELISA shows that the resulting sera react strongly with SARS-CoV-2 N-protein (FIG. 14).

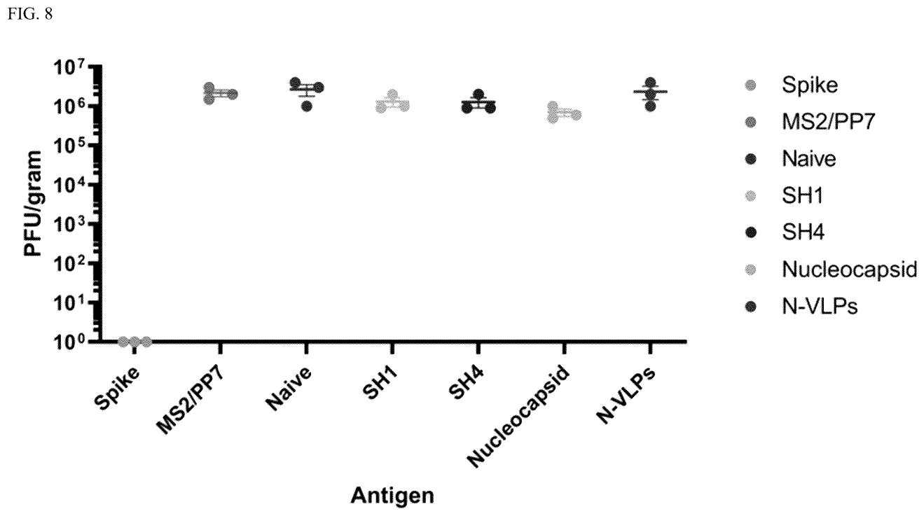

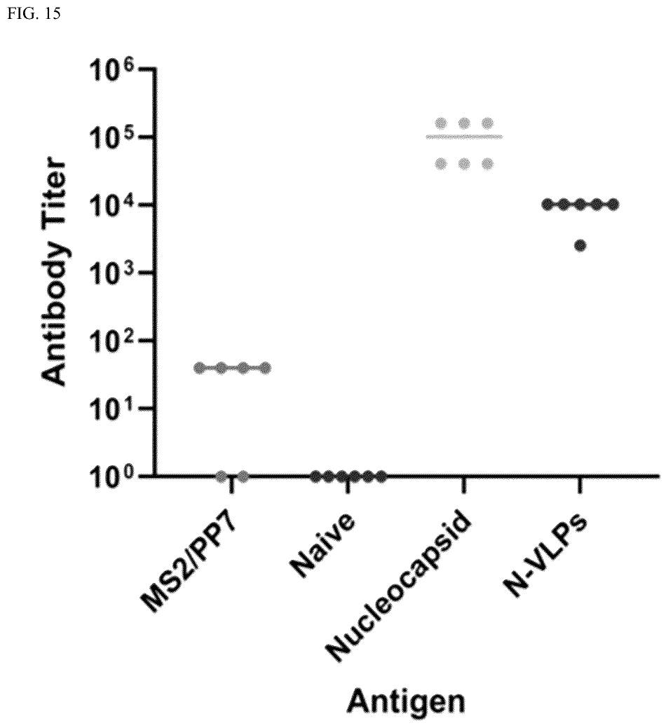

Thereafter, additional VLPs from Table 1 were prepared for immunization of hamsters. Eight VLPs from Table 1 were separately prepared and isolated, then combined to form a mixture of all eight VLPs. The VLP mixture evaluated for their ability to treat Syrian Hamsters challenged with SARS-CoV-2. Animals (nine per group) were immunized twice at three-week intervals with 10 μg of the VLP mixture. A month after the second vaccination, blood was drawn, and the sera tested by ELISA for the presence of anti-N-protein antibodies. FIG. 15 shows the end-point dilution titers. With the VLP mixture, a high-titer anti-N-protein response was obtained in each of the nine animals without the use of any adjuvant. Each VLP in the mixture can stimulate an antibody response to the specific N-protein epitope it displays, and any single VLP or combination of VLPs can be employed. Immunizing with N-protein alone (in incomplete Freund's adjuvant) yielded higher titers, presumably because of the presence of adjuvant and/or because the immunogen contains the full complement of N-protein epitopes, whereas the eight VLPs represented a total of only six epitopes and therefore produce antibodies binding only a subset of epitopes on full-length N-protein. As a positive control, animals were immunized with purified spike protein, an immunogen know to effectively prevent infection. As negative controls, one group of animals was immunized with a one-to-one mixture of MS2 and PP7 VLPs (i.e., with no foreign epitope) and another group was left untreated (naïve) (FIG. 8).

Hamsters were infected intra-nasally with SARS-CoV-2 and the course of infection was followed by daily monitoring of individual animal weights (FIG. 7). Animals immunized with spike protein (the positive control) briefly lost weight and began to recover by day 3, returning to their starting weights by about day 10. On the other hand, the negative controls (naïve animals and those immunized with MS2/PP7 VLPs) lost nearly 20% of their body weights by day 7, and by day 16 had recovered to about 95% of their starting weights. In contrast, immunization with N-protein dramatically reduced infection-associated weight loss and shortened recovery time. Strikingly, immunization with the N-epitope-specific VLPs provided effects similar to vaccination with N-protein itself.

Immunization with N-protein peptides displayed on VLPs overcomes deficiencies observed when immunizing using N-protein-targeted vaccines that deliver the complete N-protein, either as a purified protein (i.e., as a subunit vaccine) or as an N-encoding mRNA. These prior approaches typically elicit relatively short-lived antibodies. In contrast, the efficacy of a vaccine in inhibiting initial infection by SARS-CoV-2 would likely depend, at least in part, on the abundance of antibodies present at the time of infection. Thus, durability of antibody titers is desirable. The VLP platform is known to elicit high-titer and comparatively long-lived antibody responses.

### Alternative Designs

In one or more embodiments, the N-protein peptide may include one or more amino acid deletions, additions, substitutions, or a combination of such modifications to the amino acid sequence of and one of SEQ ID NO:6, SEQ ID NO:7, SEQ ID NO:8, SEQ ID NO:9, SEQ ID NO:10, SEQ ID NO: 11, SEQ ID NO:12, SEQ ID NO:13, or SEQ ID NO:14. Furthermore, additional epitopes may be designed by reference to N-protein structure. In particular, the intrinsically disordered N-terminal, C-terminal, and linker regions could be a particularly rich source of linear epitopes, whether or not they are recognized in the natural response to infection.

Regardless of the specific design, a vaccine directed against the SARS-CoV-2 nucleocapsid protein can induce new immunity in the previously uninfected or can strengthen and/or prolong existing immunity against SARS-CoV-2 in the previously infected by amplifying and prolonging the antibody response to vulnerable virus epitopes.

### Compositions

This disclosure therefore describes VLP-based immunogens that include peptides of the SARS-CoV-2 spike protein stem-helix (e.g., SEQ ID NO:1). This disclosure also describes VLP-based immunogens that include peptides of the SARS-CoV-2 nucleocapsid protein (e.g., any one of SEQ ID NOs:6-14). Further, the immunogen can include mixtures of VLPs that display more than one population of antigenic peptides—e.g., a first population of SARS-CoV-2 peptides and a second population of SARS-CoV-2 peptides. The first population of SARS-CoV-2 peptides can include peptides that include the amino acids of SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, or a structurally similar variant of any of the foregoing. The second population of SARS-CoV-2 peptides can include peptides that include the amino acids of SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, or a structurally similar variant of any of the foregoing that differs from the first population of SARS-CoV-2 peptides. Alternatively, the second population of SARS-CoV-2 peptides can include any peptides known to elicit an immune response against SARS-CoV-2. Thus, VLPs can be designed to display one, two, three, four, five, six, or more antigenic peptides.

In another aspect, an immunogenic composition may include more than one population of VLPs. For example, in one or more embodiments, an immunogenic composition can include a first population of VLPs and a second population of VLPs. The first population of VLPs can display one or more different peptides that include the amino acids of SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, or a structurally similar variant of any of the foregoing. The second population of VLPs can display one or more peptides, different from the one or more peptides displayed by the first population of VLPs, that include the amino acids of SEQ ID NO:1, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, or a structurally similar variant of any of the foregoing. Alternatively, or in addition, the second population of VLPs can display any peptide known elicit an immune response against SRAS-CoV-2.

As used herein, a peptide or variant of a peptide is “structurally similar” to a reference peptide if the amino acid sequence of the peptide possesses a specified amount of identity compared to the reference peptide. Structural similarity of two peptides can be determined by aligning the residues of the two peptides (for example, a candidate peptide and any one of SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate peptide is the peptide being compared to the reference peptide (e.g., any one of SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14). A candidate peptide can be isolated, for example, from an animal, or can be produced using recombinant techniques, or chemically or enzymatically synthesized.

A pair-wise comparison analysis of amino acid sequences can be carried out using the BESTFIT algorithm in the GCG package (version 10.2, Madison WI). Alternatively, peptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

An antigenic stem-helix peptide can include amino acids in addition to SEQ ID NO:1, so long as the additional amino acids do not eliminate immunogenicity toward the SARS-CoV-2 spike protein stem-helix. For example, an antigenic stem-helix peptide may include a linker sequence such as, for example, amino acids 23-28 of SEQ ID NO:2, amino acids 129-136 of SEQ ID NO:3, or any other suitable linker amino acid sequence known to those of skill in the art.

An antigenic N-protein peptide can include amino acids in addition to any one of SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14, so long as the additional amino acids do not eliminate immunogenicity toward the SARS-CoV-2 N-protein. For example, an antigenic N-protein peptide may include a linker sequence such as, for example, amino acids 23-28 of SEQ ID NO:2, amino acids 129-136 of SEQ ID NO:3, or any other suitable linker amino acid sequence known to those of skill in the art.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also includes the presence of conservative substitutions. A conservative substitution for an amino acid in an immunogenic peptide as described herein may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH₂. Likewise, biologically active analogs of a polypeptide containing deletions or additions of one or more contiguous or noncontiguous amino acids that do not eliminate a functional activity of the peptide are also contemplated.

In one or more embodiments, a SARS-CoV-2 stem-helix peptide as described herein can include a peptide with at least 75%, at least 80%, at least 85%, at least 90%, or at least 95% sequence similarity to the antigenic portion of the amino acids sequence of SEQ ID NO:1. That is, a SARS-CoV-2 stem-helix peptide can include a total of no more than five, no more than four, no more than three, no more than two, or no more than one amino acid deletions and non-conservative amino acid substitutions compared to the antigenic portion of SEQ ID NO:1.

The antigenic portion of SEQ ID NO:1 is the portion of SEQ ID NO:1 to which a stem-helix-specific antibody binds. Anti-stem helix mAbs characterized so far bind a shared epitope residing more or less in the middle of the 20-amino-acid sequence of SEQ ID NO:1. Thus, the antigenic portion of SEQ ID NO:1 can be a fragment having at least six contiguous amino acids of SEQ ID NO:1, at least seven contiguous amino acids of SEQ ID NO:1, at least eight contiguous amino acids of SEQ ID NO:1, at least nine contiguous amino acids of SEQ ID NO:1, at least ten contiguous amino acids of SEQ ID NO:1, at least eleven contiguous amino acids of SEQ ID NO:1, at least twelve contiguous amino acids of SEQ ID NO:1, at least thirteen contiguous amino acids of SEQ ID NO:1, at least fourteen contiguous amino acids of SEQ ID NO:1, at least fifteen contiguous amino acids of SEQ ID NO:1, at least sixteen contiguous amino acids of SEQ ID NO:1, at least seventeen contiguous amino acids of SEQ ID NO:1, at least eighteen contiguous amino acids of SEQ ID NO:1, at least nineteen contiguous amino acids of SEQ ID NO:1, or all twenty amino acids of SEQ ID NO:1. The antigenic portion of SEQ ID NO:1 may start with any amino acid residue so that the fragment includes the middle residues of SEQ ID NO: 1. Thus, for example, the antigenic portion of SEQ ID NO:1 may include, but is not limited to, a fragment that includes amino acids 3-10 of SEQ ID NO:1, amino acids 7-17 of SEQ ID NO:1, amino acids 4-13 of SEQ ID NO:1, amino acids 4-15 of SEQ ID NO:1, amino acids 4-16 of SEQ ID NO:1, amino acids 5-12 of SEQ ID NO:1, amino acids 5-13 of SEQ ID NO:1, amino acids 5-14 of SEQ ID NO:1, amino acids 5-15 of SEQ ID NO:1, amino acids 6-13 of SEQ ID NO:1, amino acids 6-14 of SEQ ID NO:1, amino acids 6-18 of SEQ ID NO:1, amino acids 7-14 of SEQ ID NO:1, amino acids 7-15 of SEQ ID NO:1, amino acids 7-17 of SEQ ID NO:1, amino acids 8-15 of SEQ ID NO:1, amino acids 8-16 of SEQ ID NO:1, amino acids 9-15 of SEQ ID NO:1, amino acids 9-17 of SEQ ID NO:1, or amino acids 10-17 of SEQ ID NO:1.

In one or more embodiments, a SARS-CoV-2 stem-helix peptide as described herein can include a peptide with a segment having at least at least 75%, at least 80%, at least 85%, at least 90%, or at least 95% sequence identity the antigenic portion of the amino acids sequence of SEQ ID NO:1. That is, a SARS-CoV-2 stem-helix peptide can include a segment having a total of no more than five, no more than four, no more than three, no more than two, or no more than one amino acid deletions and amino acid substitutions compared to the antigenic portion of SEQ ID NO:1.

In one or more embodiments, a SARS-CoV-2 N-protein peptide as described herein can include a peptide with a segment having at least 76%, at least 80%, at least 82%, at least 85%, at least 88%, at least 90%, at least 94%, or at least 95% sequence similarity to antigenic portion of the amino acid sequence of any one of SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14. That is, a SARS-CoV-2 N-protein peptide can include a segment having a total of no more than four, no more than three, no more than two, or no more than one amino acid deletions and non-conservative amino acid substitutions compared to any one of SEQ ID NO:6, SEQ ID NO:7, SEQ ID NO:8, SEQ ID NO:9, SEQ ID NO:10, SEQ ID NO: 11, SEQ ID NO:12, SEQ ID NO:13, or SEQ ID NO:14.

In one or more embodiments, a SARS-CoV-2 N-protein peptide as described herein can include a peptide with a segment having at least at least 76%, at least 80%, at least 82%, at least 85%, at least 88%, at least 90%, at least 94%, or at least 95% sequence identity to any one of SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14. That is, a SARS-CoV-2 N-protein peptide can include a segment having a total of no more than four, no more than three, no more than two, or no more than one amino acid deletions and amino acid substitutions compared to any one of SEQ ID NO:6, SEQ ID NO:7, SEQ ID NO:8, SEQ ID NO:9, SEQ ID NO:10, SEQ ID NO:11, SEQ ID NO:12, SEQ ID NO:13, or SEQ ID NO:14.

In one or more embodiments, a stem-helix peptide or an N-protein peptide as described herein can be designed to provide additional sequences, such as, for example, the addition of added C-terminal or N-terminal amino acids that can, for example, facilitate purification by trapping on columns or use of antibodies. Such tags include, for example, histidine-rich tags that allow purification of polypeptides on nickel columns. Such modification techniques and suitable additional sequences are well known in the molecular biology arts.

The virus-like particle (VLP) can include any particle that includes viral proteins assembled to structurally resemble the virus from which they are derived but lack enough of the viral genome so that they are non-replicative and, therefore, noninfectious. A VLP may, therefore, include at least some of the viral genome, but the viral genome is genetically modified so that the viral genes responsible for infectivity and replication are inactivated. Exemplary VLPs include, but are not limited to, VLPs of QP, MS2, PP7, AP205, or other bacteriophage coat proteins, the capsid and core proteins of Hepatitis B virus, measles virus, Sindbis virus, rotavirus, foot-and-mouth-disease virus, Norwalk virus, the retroviral GAG protein, the retrotransposon Ty protein pl, the surface protein of Hepatitis B virus, human papilloma virus, human polyoma virus, RNA phages, Ty, fr-phage, GA-phage, AP 205-phage and, in particular, Qp-phage, Cowpea chlorotic mottle virus, cowpea mosaic virus, human papilloma viruses (HPV), bovine papilloma viruses, porcine parvovirus, parvoviruses such as B19, porcine (PPV) and canine (CPV) parvovirues, caliciviruses (e.g. Norwalk virus, rabbit hemorrhagic disease virus [RHDV]), animal hepadnavirus core Antigen VLPs, filamentous/rod-shaped plant viruses, including but not limited to Tobacco Mosaic Virus (TMV), Potato Virus X (PVX), Papaya Mosaic Virus (PapMV), Alfalfa Mosaic Virus (AIMV), and Johnson Grass Mosaic Virus (JGMV), insect viruses such as flock house virus (FHV) and tetraviruses, polyomaviruses such as Murine Polyomavirus (MPyV), Murine Pneumotropic Virus (MPtV), BK virus (BKV), and JC virus (JCV).

The antigenic peptides may be coupled to immunogenic carriers via chemical conjugation or by expression of genetically engineered fusion partners. In this context, the term “antigenic peptide” refers, collectively to a stem-helix peptide as described herein, a nucleocapsid (N) protein as described herein, or any other peptide known to elicit an immunogenic response against SARS-CoV-2. In the plural “antigenic peptides” refers to any combination of two or more antigenic peptides—e.g., a stem-helix peptide and an N-protein peptide; a stem-helix peptide and a peptide known to elicit an immunogenic response against SARS-CoV-2; two N-protein peptides; an N-protein peptide and a peptide known to elicit an immunogenic response against SARS-CoV-2; a stem-helix peptide, an N-protein peptide, and a peptide known to elicit an immunogenic response against SARS-CoV-2; a stem-helix peptide and two N-protein peptides, etc.

The coupling does not necessarily need to be direct but can occur through linker sequences. More generally, in the case that antigenic peptides either fused, conjugated, or otherwise attached to an immunogenic carrier, spacer sequence, or linker sequence are typically added at one or both ends of the antigenic peptides. The coupling approach used for any one antigenic peptide may be selected independently of the coupling approach used for any other antigenic peptide.

In one embodiment, the antigenic peptide may be displayed as fusion protein with a subunit of the immunogenic carrier. Fusion of the peptide can be effected by inserting the antigenic peptide amino acid sequence into the immunogenic carrier primary sequence, or by fusion to either the N-terminus or C-terminus of the immunogenic carrier.

When the immunogenic carrier is a VLP, the chimeric antigenic peptide-VLP subunit can be capable of self-assembly into a VLP. VLP displaying epitopes fused to their subunits are also herein referred to as chimeric VLPs. For example, European Application No. EP90310264A (European Patent No. EP0421635 B1) describes the use of chimeric hepadnavirus core antigen particles to present foreign peptide sequences in a virus-like particle.

Flanking amino acid residues may be added to either end of the sequence of the antigenic peptide to be fused to either end of the sequence of the subunit of a VLP, or for internal insertion of such peptide sequence into the sequence of the subunit of a VLP. Glycine and serine residues are particularly favored amino acids to be used in the flanking sequences added to the peptide to be fused. Glycine residues confer additional flexibility, which may diminish the potentially destabilizing effect of fusing a foreign sequence into the sequence of a VLP subunit.

In one or more embodiments, the immunogenic carrier is a VLP of a RNA phage, preferably MS2, PP7, or Q13. The major coat proteins of RNA phages spontaneously assemble into VLPs upon expression in bacteria such as, for example, E. coli.

Further VLPs suitable for fusion of antigens or antigenic determinants are described in, for example, International Patent Application No. PCT/IB2002/004132 (International Publication No. WO 03/024481 A2) and include bacteriophage fr, capsid protein of papillomavirus, retrotransposon Ty, yeast and also Retrovirus-like-particles, HIV2 Gag, Cowpea Mosaic Virus, parvovirus VP2 VLP, HBsAg (U.S. Pat. No. 4,722,840). Examples of chimeric VLPs suitable for use as the immunogenic carrier include those described in Kozlovska et al., 1996, Intervirology 39:9-15. Further examples of VLPs suitable for use as the immunogenic carrier include, but are not limited to, HPV-1, HPV-6, HPV-11, HPV-16, HPV-18, HPV-33, HPV-45, CRPV, COPV, HIV GAG, Tobacco Mosaic Virus, Virus-like particles of SV-40, Polyomavirus, Adenovirus, Herpes Simplex Virus, Rotavirus, and Norwalk virus.

In a preferred embodiment, the stem-helix peptide of SEQ ID NO:1 is fused to the N-terminus of the MS2 coat protein (SEQ ID NO:2). In another preferred embodiment, the stem-helix peptide of SEQ ID NO:1 is fused to the C-terminus of the PP7 coat protein (SEQ ID NO:3).

For any recombinantly expressed antigenic peptide described herein (whether or not coupled to an immunogenic carrier), this disclosure describes the nucleic acid that encodes the peptide or protein, an expression vector containing the nucleic acid, and a host cell containing the expression vector (autonomously or chromosomally inserted). This disclosure further describes a method of recombinantly producing the peptide or protein by expressing it in a host cell, with or without further isolating the immunogen.

Thus, this disclosure describes an isolated nucleic acid sequence that encodes any embodiment of an antigenic peptide, whether a stem-helix peptide or an N-protein peptide, described herein. In one or more embodiments, the isolated nucleic acid encodes the antigenic peptide of SEQ ID NO:1, SEQ ID NO:6, SEQ ID NO:7, SEQ ID NO:8, SEQ ID NO:9, SEQ ID NO:10, SEQ ID NO:11, SEQ ID NO:12, SEQ ID NO: 13, SEQ ID NO:14, or a structurally similar variant of any of the foregoing. Given the amino acid sequence of any antigenic peptide, a person of ordinary skill in the art can determine the full scope of polynucleotides that encode that amino acid sequence using conventional, routine methods.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugates, and oligonucleotides. A nucleic acid may be single-stranded, double-stranded, linear, or covalently circularly closed molecule. A nucleic acid can be isolated. The term “isolated nucleic acid” means that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be introduced—i.e., transfected-into cells. When RNA is used to transfect cells, the RNA may be modified by stabilizing modifications, capping, or polyadenylation.

As used herein “amplified DNA” or “PCR product” refers to an amplified fragment of DNA of defined size. Various techniques are available and well known in the art to detect PCR products. PCR product detection methods include, but are not restricted to, gel electrophoresis using agarose or polyacrylamide gel and adding ethidium bromide staining (a DNA intercalant), labeled probes (radioactive or non-radioactive labels, southern blotting), labeled deoxyribonucleotides (for the direct incorporation of radioactive or non-radioactive labels) or silver staining for the direct visualization of the amplified PCR products; restriction endonuclease digestion, which relies on agarose gel electrophoresis, polyacrylamide gel electrophoresis, or high-performance liquid chromatography (HPLC); dot blots, using the hybridization of the amplified DNA on specific labeled probes (radioactive or non-radioactive labels); high-pressure liquid chromatography using ultraviolet detection; electro-chemiluminescence coupled with voltage-initiated chemical reaction/photon detection; and direct sequencing using radioactive or fluorescently labeled deoxyribonucleotides for the determination of the precise order of nucleotides with a DNA fragment of interest, oligo ligation assay (OLA), PCR, qPCR, DNA sequencing, fluorescence, gel electrophoresis, magnetic beads, allele specific primer extension (ASPE) and/or direct hybridization.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; and 9,012,208. Examples of nucleic acid analysis include, but are not limited to, sequencing and DNA-protein interaction. Sequencing may be by any method known in the art. DNA sequencing techniques include classic dideoxy sequencing reactions (Sanger method) using labeled terminators or primers and gel separation in slab or capillary, and next generation sequencing methods such as sequencing by synthesis using reversibly terminated labeled nucleotides, pyrosequencing, 454 sequencing, Illumina/Solexa sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes.

This disclosure also describes a host cell including any of the isolated nucleic acid sequences and/or antigenic peptides described herein. Thus, this disclosure encompasses translation of a nucleic acid (e.g., an mRNA) by a host cell to produce any embodiment of antigenic peptide disclosed herein and/or any embodiment of VLP cribbed herein that displays any one or more antigenic peptide.

The nucleic acid constructs of the present invention may be introduced into a host cell to be altered, thus allowing expression within the cell of the antigenic peptide and/or the VLP displaying any one or more antigenic peptides, thereby generating a genetically engineered cell. A variety of methods are known in the art and suitable for introducing a nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE (Thermo Fisher Scientific, Inc., Waltham, MA), HILYMAX (Dojindo Molecular Technologies, Inc., Rockville, MD), FUGENE (Promega Corp., Madison, WI), JETPEI (Polyplus Transfection, Illkirch, France), EFFECTENE (Qiagen, Hilden, Germany) and DreamFect (OZ Biosciences, Inc USA, San Diego, CA).

The nucleic acid constructs described herein may be introduced into a host cell to be altered, thus allowing expression within the cell of the protein encoded by the nucleic acid. A variety of host cells are known in the art and suitable for protein expression. Examples of typical cell used for transfection and protein expression include, but are not limited to, a bacterial cell, a eukaryotic cell, a yeast cell, an insect cell, or a plant cell such as, for example, E. coli, Bacillus, Streptomyces, Pichia pastoris, Salmonella typhimurium, Drosophila S2, Spodoptera SJ9, CHO, COS (e.g., COS-7),3T3-F442A, HeLa, HUVEC, HUAEC, NIH 3T3, Jurkat, 293, 293H, or 293F.

In one or more embodiments, the antigenic peptide can be chemically coupled to the immunogenic carrier using techniques well known in the art. Conjugation can occur to allow free movement of peptides via single point conjugation (e.g., either N-terminal or C-terminal point) or as a locked down structure where both ends of peptides are conjugated to either an immunogenic carrier protein or to a scaffold structure such as a VLP. Conjugation occurs via conjugation chemistry known to those skilled in the art such as via cysteine residues, lysine residues, or another carboxy moiety. Thus, for example, for direct covalent coupling, it is possible to use a carbodiimide, glutaraldehyde, or N-[y-maleimidobutyryloxy] succinimide ester, using common commercially available hetero-bifunctional linkers such as 1-cyano-4-dimethylaminopyridinium tetrafluoroborate (CDAP) or succinimidyl 3-(2-pyridyldithio)propionate (SPDP).

Examples of conjugation of peptides, particularly cyclized peptides, to a protein carrier via acylhydrazine peptide derivatives are described in, for example, International Patent Application No. PCT/EP2003/004551 (International Publication No. WO 2003/092714 A1). After the coupling reaction, the immunogen can easily be isolated and purified using, for example, a dialysis method, a high-performance liquid chromatography method, a gel filtration method, a fractionation method, etc. Peptides terminating with a cysteine residue (preferably with a linker outside the cyclized region) may be conveniently conjugated to a carrier protein via maleimide chemistry.

Several antigenic peptides, either having an identical amino acid sequence or different amino acid sequences, may be coupled to a single VLP particle, leading preferably to a repetitive and ordered structure presenting several antigenic determinants in an oriented manner as described in International Patent Applications PCT/IB1999/001925 (International Publication No. WO 00/032227), PCT/IB2002/004132 (International Publication No. WO 2003/024481), PCT/IB2002/000166 (International Publication No. WO 02/056905), and PCT/EP2003/007572 (International Publication No. WO 2004/007538). Thus, the antigenic peptide displayed by one VLP subunit in a VLP may the same or different than the antigenic peptide displayed by a second VLP subunit in the same VLP. In other embodiments, one or several antigen molecules can be attached to one VLP subunit. A specific feature of the VLP of the coat protein of RNA phages is thus the possibility to couple several antigens per subunit. This allows for the generation of a dense antigen array.

Another feature of VLPs derived from RNA phage is their high expression yield in bacteria that allows production of large quantities of material at affordable cost. Moreover, the use of the VLPs as carriers allows the formation of robust antigen arrays and conjugates, respectively, with variable antigen density. In particular, the use of VLPs of RNA phages allows a very high antigen density to be achieved.

### Methods of Treatment

VLPs displaying one or more antigenic peptides may be used to treat a subject having, or at risk of having, any condition caused by infection by SARS-CoV-2 such as, but not limited to, COVID-19.

As used herein, “treat” or variations thereof refer to reducing, limiting progression, ameliorating, or resolving, to any extent, the symptoms or signs related to a condition. A “sign” or “clinical sign” refers to an objective physical finding relating to a particular condition capable of being found by one other than the patient. A “symptom” refers to any subjective evidence of disease or of a patient's condition.

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject.

Treatment that is prophylactic—e.g., initiated before a subject manifests a symptom or clinical sign of the condition such as, for example, while a tumor remains subclinical—is referred to herein as treatment of a subject that is “at risk” of having the condition. As used herein, the term “at risk” refers to a subject that may or may not actually possess the described risk. Thus, for example, a subject “at risk” of developing a condition is a subject possessing one or more risk factors associated with the condition such as, for example, genetic predisposition, ancestry, age, sex, geographical location, lifestyle, or medical history. Thus, the VLP displaying one or more antigenic peptides may be administered before a subject manifests a symptom or clinical sign of infection by SARS-CoV-2.

Accordingly, a composition can be administered before, during, or after the subject first exhibits a symptom or clinical sign of the condition caused by infection by SARS-CoV-2 (e.g., COVID-19). Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

Thus, the method includes administering an effective amount of the composition to a subject having, or at risk of having, a condition characterized, at least in part, by infection by SARS-CoV-2. In this aspect, an “effective amount” is an amount effective to reduce, limit progression, ameliorate, or resolve, to any extent, a symptom or clinical sign related to the condition.

Thus, any embodiment of the VLPs described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the VLP without causing any undesirable biological effects or interacting in a deleterious manner with any of the other components of the pharmaceutical composition in which it is contained.

The VLP may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, a VLP may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the VLP into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The amount of VLP administered can vary depending on various factors including, but not limited to, the cancer being treated, the weight, physical condition, and/or age of the subject, and/or the route of administration. Thus, the absolute weight of VLP included in a given unit dosage form can vary widely, and depends upon factors such as the species, age, weight, and physical condition of the subject, and/or the method of administration. Accordingly, it is not practical to set forth generally the amount that constitutes an amount of VLP effective for all possible applications. Those of ordinary skill in the art, however, can readily determine the appropriate amount with due consideration of such factors.

In one or more embodiments, the method can include administering sufficient VLP to provide a dose of, for example, from about 50 ng/kg to about 1 mg/kg to the subject, although in one or more embodiments the methods may be performed by administering VLP in a dose outside this range.

In one or more embodiments, the method includes administering sufficient VLP to provide a minimum dose of at least 50 ng/kg such as, for example, at least 100 ng/kg, at least 200 ng/kg, at least 300 ng/kg, at least 400 ng/kg, at least 500 ng/kg, at least 600 ng/kg, at least 700 ng/kg, at least 800 ng/kg, at least 900 ng/kg, at least 1 μg/kg, at least 2 μg/kg, at least 5 μg/kg, at least 10 μg/kg, at least 20 μg/kg, at least 50 μg/kg, at least 100 μg/kg, at least 200 μg/kg, or at least 500 μg/kg.

In one or more embodiments, the method includes administering sufficient VLP to provide a maximum dose of no more than 1 mg/kg, no more than 500 μg/kg, no more than 250 μg/kg, no more than 200 μg/kg, no more than 150 μg/kg, no more than 100 μg/kg, no more than 50 μg/kg, no more than 25 μg/kg, no more than 10 μg/kg, no more than 5 μg/kg, no more than 2 μg/kg, no more than 1 μg/kg, no more than 800 ng/kg, no more than 600 ng/kg, no more than 500 ng/kg, no more than 400 ng/kg, no more than 300 ng/kg, no more than 250 ng/kg, no more than 150 ng/kg, no more than 100 ng/kg, no more than 50 ng/kg, or no more than 25 ng/kg.

In one or more embodiments, the method includes administering sufficient VLP to provide a dose that falls within a range having as endpoints any minimum dose listed above and any maximum dose listed above that is greater than the minimum does. For example, in one or more embodiments, the method can includes administering sufficient VLP to provide a dose of from 200 ng/kg to about 10 μg/kg to the subject, for example, a dose of from about 700 ng/kg to about 5 μg/kg.

In one or more embodiments, VLP may be administered, for example, from a single dose to multiple doses per week, although in one or more embodiments the method can be performed by administering VLP at a frequency outside this range. When multiple doses are used within a certain period, the amount of each dose may be the same or different. For example, a dose of 1 mg per day may be administered as a single dose of 1 mg, two 0.5 mg doses, or as a first dose of 0.75 mg followed by a second dose of 0.25 mg. Also, when multiple doses are used within a certain period, the interval between doses may be the same or be different.

In certain embodiments, VLP may be administered at minimum frequency of at least once per year such as, for example, at least once every six months, at least once every four months, at least once every three months, at least once every two months, at least once per month, or at least once every two weeks.

In certain embodiments, VLP may be administered at maximum frequency of no more than once per week such as, for example, no more than once every two weeks, no more than once per month, no more than once every two months, no more than once every three months, no more than once every six months, or once per year.

In one or more embodiments, VLP may be administered at a frequency defined by a range having as endpoints any minimum frequency listed above and any maximum frequency listed above that is more frequent than the minimum frequency.

The duration of administration of an antigenic VLP described herein, e.g., the period of time over which an antigenic VLP is administered, can vary, depending on any of a variety of factors, e.g., patient response, etc. For example, an antigenic VLP can be administered over a period of time ranging from about one day to about one week, from about two weeks to about four weeks, from about one month to about two months, from about two months to about four months, from about four months to about six months, from about six months to about eight months, from about eight months to about one year, from about one year to about two years, or from about two years to about four years, or more. In one or more embodiments, the VLP may be administered as a once off treatment. In other embodiments, the VLP may be administered for the life of the subject. In certain embodiments, the VLP may be administered monthly (or every four weeks) until effective.

In one or more embodiments, the VLP may be administered at an initial frequency for an initial period and then administered at a lower frequency thereafter. For example, a dosing regimen may include administering three doses of the VLP at a frequency of once per month (i.e., an initial dose followed by a second dose one month after the initial dose) followed by an additional dose six months after the initial dose.

When a VLP composition is used for prophylactic treatment, it may be generally administered for priming and/or boosting doses. Boosting doses, when administered, are adequately spaced (e.g., yearly) to boost the level of circulating antibody that has fallen below a desired level. Boosting doses may include an antigenic peptide either with or in the absence of the original immunogenic carrier. A booster composition may include an alternative immunogenic carrier or may be in the absence of any carrier. Moreover, a booster composition may be formulated either with or without adjuvant.

In some cases, the method can further include administering to the subject an additional therapeutic agent effective for treating the condition (e.g., COVID-19). For example, therapy involving the VLP may be combined with conventional therapies for COVID-19. As another example, the VLP compositions described herein can increase efficacy of an existing SARS-CoV-2 vaccine.

### EXAMPLES

The present invention is illustrated by the following examples. It is to be understood that the particular examples, materials, amounts, and procedures are to be interpreted broadly in accordance with the scope and spirit of the invention as set forth herein.

### Construction of Stem-Helix VLPs

The SARS-CoV-2 stem-helix peptide LQPELDSFKEELDKYFKNHT (SEQ ID NO:1) was reverse translated it (with E. coli codon preferences) and then incorporated it into PCR primers suitable for fusion to the MS2 coat protein N-terminus or the PP7 coat protein C-terminus. When paired with appropriate primers annealing to the other ends of the MS2 and PP7 sequences, PCR generated coding sequences for the two coat protein/stem-helix fusions SEQ ID NO:2 and SEQ ID NO:3. The PCR products, flanked with NcoI and BamHI sites, were then cloned in plasmid pDSP62 (Chackerian et al., 2011, J. Mol. Biol. 409:225-237) under T7 promoter control and expressed in E. coli. SEQ ID NO:2, sometimes referred to herein as the SH1 construct, has the 20-amino-acid stem-helix peptide fused to MS2 coat protein N-terminus through a six-amino-acid linker sequence. SEQ DI NO:3, sometimes referred to herein as the SH4 construct, has the 20-amino-acid stem-helix peptide fused to the PP7 coat protein C-terminus through an eight-amino-acid linker.

### Bacterial Production of the Stem-Helix VLPs

The DNA sequences shown above are flanked by NeoI and BamHI sites, which makes them easy to insert in place of the MS2 coat protein single-chain dimer sequences in pDSP62 (Chackerian et al., 2011, J. Mol. Biol. 409:225-237), putting them under control of the T7 promoter. The plasmids were introduced into chemically competent cells (OVEREXPRESS C41(DE3), Sigma-Aldrich, Merck KGaA, Darmstadt, Germany), which were then grown in auto-inducing medium overnight at 30 C. The VLPs were extracted from cells and purified by methods previously described (Peabody et al., 2008, J. Mol. Biol. 380:252-263).

### Construction of N-Protein VLPs

The sequences of peptide epitopes recognized by Covid-19 patient's antibodies were identified by deep sequence analysis (ION TORRENT, Thermo Fisher Scientific, Inc., Waltham, MA) of the population of peptides enriched by affinity-selection from an antigen fragment library. This process yields sequence information for thousands of clones simultaneously but does not directly yield any clones from which a VLP can be produced. Thus, it was necessary to synthesize oligonucleotide primers that fused a desired peptide sequence to MS2 coat protein. Specifically, in each case an oligonucleotide primer was synthesized that allows insertion of an appropriate epitope-encoding sequence into the AB-loop of the MS2 single-chain dimer. Again, the construction of VLP clones was necessary because deep sequence analysis yields many thousands of individual sequences, but not the corresponding clones themselves. Had we instead used, say, Sanger sequencing, we would have first isolated individual clones and then sequenced each of them individually. Those clones could be used directly for VLP synthesis without the need of constructing from knowledge of the sequence.

### Bacterial Production of N-Protein VLPs

Plasmids encoding each VLP were introduced by electroporation into E. coli (usually strain C41(DE3)) and VLP synthesis was induced with IPTG in cultures grown to mid-log phase and then grown an additional five hours. Alternatively, some cultures were grown in auto-inducing medium overnight. Cells were collected by centrifugation, lysed by sonication and VLPs purified by gel filtration chromatography as described previously.

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein, the word “exemplary” means to serve as an illustrative example and should not be construed as preferred or advantageous over other embodiments.

The words “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

### Sequence Listing Free Text

-SARS-CoV-2 spike protein stem-helix peptide
SEQ ID NO: 1
LQPELDSFKE ELDKYFKNHT
-Stem-helix-MS2 fusion (SH1)
SEQ ID NO: 2
Met Ala Leu Gln Pro Glu Leu Asp Ser Phe Lys Glu Glu Leu Asp Lys Tyr Phe Lys Asn
His Thr Ser Thr Gly Val Gly Ser Ala Ser Asn Phe Thr Gln Phe Val Leu Val Asp Asn
Gly Gly Thr Gly Asp Val Thr Val Ala Pro Ser Asn Phe Ala Asn Gly Val Ala Glu Trp
Ile Ser Ser Asn Ser Arg Ser Gln Ala Tyr Lys Val Thr Cys Ser Val Arg Gln Ser Ser
Ala Gln Asn Arg Lys Tyr Thr Ile Lys Val Glu Val Pro Lys Val Ala Thr Gln Thr Val
Gly Gly Val Glu Leu Pro Val Ala Ala Trp Arg Ser Tyr Leu Asn Met Glu Leu Thr Ile
Pro Ile Phe Ala Thr Asn Ser Asp Cys Glu Leu Ile Val Lys Ala Met Gln Gly Leu Leu
Lys Asp Gly Asn Pro Ile Pro Ser Ala Ile Ala Ala Asn Ser Gly Ile Tyr End End Thr
Pro Gly
Stem-Helix 20-mer peptide: amino acids 3-22
Linker: amino acids 23-28
-PP7-Stem-helix fusion (SH4)
SEQ ID NO: 3
Met Ala Lys Thr Ile Val Leu Ser Val Gly Glu Gly Thr Arg Thr Leu Thr Glu Ile Gln
Ser Thr Ala Asp Arg Gln Ile Phe Glu Glu Lys Val Gly Pro Leu Val Gly Arg Leu Arg
Leu Thr Ala Ser Leu Arg Gln Asn Gly Ala Lys Thr Ala Tyr Arg Val Asn Leu Lys Leu
Asp Gln Ala Asp Val Val Asp Cys Ser Thr Ser Val Cys Gly Glu Leu Pro Lys Val Arg
Tyr Thr Gln Val Trp Ser His Asp Val Thr Ile Val Ala Asn Ser Thr Glu Ala Ser Arg
Lys Ser Leu Tyr Asp Leu Thr Lys Ser Leu Val Ala Thr Ser Gln Val Glu Asp Leu Val
Val Asn Leu Val Pro Leu Gly Arg Gly Gly Pro Ser Glu Ser Gly Ala Leu Gln Pro Glu
Leu Asp Ser Phe Lys Glu Glu Leu Asp Lys Tyr Phe Lys Asn His Thr
Linker: amino acids 129-136
Stem-Helix 20-mer peptide: amino acids 137-156
-Nucleotide sequence encoding SH1
SEQ ID NO: 4
ATGGCTCTGC AGCCGGAACT GGACTCTTTC AAAGAAGAAC TGGACAAATA CTTCAAAAAC CACACCTCAA
CCGGAGTTGG AAGCGCTTCT AACTTTACTC AGTTCGTTCT CGTCGACAAT GGCGGAACTG GCGACGTGAC
TGTCGCCCCA AGCAACTTCG CTAACGGGGT CGCTGAATGG ATCAGCTCTA ACTCGCGTTC ACAGGCTTAC
AAAGTAACCT GTAGCGTTCG TCAGAGCTCT GCGCAGAATC GCAAATACAC CATCAAAGTC GAGGTGCCTA
AAGTGGCAAC CCAGACTGTT GGTGGTGTAG AGCTTCCTGT AGCCGCATGG CGTTCGTACT TAAATATGGA
ACTAACCATT CCAATTTTCG CTACGAATTC CGACTGCGAG CTTATTGTTA AGGCAATGCA AGGTCTCCTA
AAAGATGGAA ACCCGATTCC CTCAGCAATC GCAGCAAACT CCGGCATCTA CTAATAGACG CCGGGTTAA
-Nucleotide sequence encoding SH4
SEQ ID NO: 5
ATGGCCAAAA CCATCGTTCT TTCGGTCGGC GAGGGTACCC GCACTCTGAC TGAGATCCAG TCCACCGCAG
ACCGTCAGAT CTTCGAAGAG AAGGTCGGGC CTCTGGTGGG TCGGCTGCGC CTCACGGCTT CGCTCCGTCA
AAACGGAGCC AAGACCGCGT ATCGAGTCAA CCTAAAACTG GATCAGGCGG ACGTCGTTGA TTGCTCCACC
AGCGTCTGCG GCGAGCTTCC GAAAGTGCGC TACACTCAGG TATGGTCGCA CGACGTGACA ATCGTTGCGA
ATAGCACCGA GGCCTCGCGC AAATCGTTGT ACGATTTGAC CAAGTCCCTC GTCGCGACCT CGCAGGTCGA
AGATCTTGTC GTCAACCTTG TGCCGCTGGG CCGTGGTGGT CCGTCTGAAT CTGGTGCTCT GCAGCCGGAA
CTGGACTCTT TCAAAGAAGA ACTGGACAAA TACTTCAAAA ACCACACCTA A
-NP1 amino acid sequence
SEQ ID NO: 6
KQRRPQGLPN NTASWFT
-NP2 amino acid sequence
SEQ ID NO: 7
LPKGFYAEGS
-NP3 amino acid sequence
SEQ ID NO: 8
RIRGGDGKMK DL
-NP4 amino acid sequence
SEQ ID NO: 9
KMSGKGQQQQ GQT
-NP5 amino acid sequence
SEQ ID NO: 10
KQRRPQGLPN NT
-NP6 amino acid sequence
SEQ ID NO: 11
RRPQGLPNNT AS
-Nuc1 amino acid sequence
SEQ ID NO: 12
EDLKFPRGQG V
-Nuc2 amino acid sequence
SEQ ID NO: 13
PQGTTLPKGF
-Nuc3 amino acid sequence
SEQ ID NO: 14
ADLDDFSKQL Q

What is claimed is:

1. An immunogen comprising:
an immunogenic carrier comprising an RNA bacteriophage virus-like particle (VLP); and
an antigenic SARS-CoV-2 stem-helix peptide linked to the immunogenic carrier.

2. The immunogen of claim 1, wherein the RNA VLP comprises MS2 bacteriophage.

3. The immunogen of claim 1, wherein the RNA VLP comprises PP7 bacteriophage.

4. The immunogen of claim 1, wherein the SARS-CoV-2 stem-helix peptide comprises a segment having at least 75% sequence similarity to an antigenic portion of SEQ ID NO:1.

5. A composition comprising the immunogen of claim 1.

6. An immunogen comprising:
an immunogenic carrier comprising an RNA bacteriophage virus-like particle (VLP); and
an antigenic SARS-CoV-2 nucleocapsid (N) protein peptide linked to the immunogenic carrier.

7. The immunogen of claim 6, wherein the RNA VLP comprises MS2 bacteriophage.

8. The immunogen of claim 6, wherein the antigenic SARS-CoV-2 N-protein peptide comprises a segment having at least 75% sequence similarity to any one of SEQ ID NOs:6-14.

9. A composition comprising the immunogen of claim 6.

10. The composition of claim 5, comprising:
a first population of VLPs displaying an antigenic SARS-CoV-2 stem-helix peptide; and
a second population of VLPs displaying an antigenic SARS-CoV-2 peptide that differs from the SARS-CoV-2 stem-helix peptide displayed by the first population of VLPs.

11. The composition of claim 10, wherein the SARS-CoV-2 stem-helix peptide comprises a segment having at least 75% sequence similarity to SEQ ID NO:1.

12. The composition of claim 10, wherein the antigenic SARS-CoV-2 peptide displayed by the second population of VLPs comprises an antigenic SARS-CoV-2 N-protein peptide comprises a segment having at least 75% sequence similarity to any one of SEQ ID NOs:6-14.

13. The composition of claim 10, further comprising an adjuvant.

14. The composition of claim 9, comprising:
a first population of VLPs displaying an antigenic SARS-CoV-2 N-protein peptide; and
a second population of VLPs displaying an antigenic SARS-CoV-2 N-protein peptide that differs from the SARS-CoV-2 N-protein peptide displayed by the first population of VLPs.

15. A method of increasing serum titer of anti-SARS-CoV-2 antibodies in a subject, the method comprising administering to the subject a therapeutically effective amount of a composition, the composition comprising:
an immunogenic carrier comprising an RNA bacteriophage virus-like particle (VLP); and
an antigenic SARS-CoV-2 stem-helix peptide linked to the immunogenic carrier.

16. The method of claim 15, wherein the SARS-CoV-2 stem-helix peptide comprises a segment having at least 75% sequence similarity to SEQ ID NO:1.

17. The method of claim 15, wherein the composition further comprises:
a second population of immunogenic carriers comprising an RNA bacteriophage virus-like particle (VLP); and
an antigenic SARS-CoV-2 peptide linked to the immunogenic carriers in the second population of immunogenic carriers, wherein the antigenic SARS-CoV-2 peptide differs from the SARS-CoV-2 stem-helix peptide.

18. The method of claim 15, the method further comprises administering to the individual at least one additional therapeutic agent for treating infection by SARS-CoV-2.

19. The method of claim 15, wherein the composition is administered to the individual before the individual manifests a symptom or clinical sign of infection by SARS-CoV-2.

20. The method of claim 15, wherein the composition is administered to the individual after the individual manifests a symptom or clinical sign of infection by SARS-CoV-2.

21. A method of increasing serum titer of anti-SARS-CoV-2 antibodies in a subject, the method comprising administering to the subject a therapeutically effective amount of a composition, the composition comprising:
an immunogenic carrier comprising an RNA bacteriophage virus-like particle (VLP); and
an antigenic SARS-CoV-2 nucleocapsid (N) protein peptide linked to the immunogenic carrier.

22. The method of claim 21, wherein the SARS-CoV-2 N-protein peptide comprises a segment having at least 75% sequence similarity to any one of SEQ ID NOs:6-14.

23. The method of claim 21, wherein the composition further comprises:
a second population of immunogenic carriers comprising an RNA bacteriophage virus-like particle (VLP); and
an antigenic SARS-CoV-2 peptide linked to the immunogenic carriers in the second population of immunogenic carriers, wherein the antigenic SARS-CoV-2 peptide differs from the SARS-CoV-2 N-protein peptide.

24. The method of claim 21, the method further comprises administering to the individual at least one additional therapeutic agent for treating infection by SARS-CoV-2.

25. The method of claim 21, wherein the composition is administered to the individual before the individual manifests a symptom or clinical sign of infection by SARS-CoV-2.

26. The method of claim 21, wherein the composition is administered to the individual after the individual manifests a symptom or clinical sign of infection by SARS-CoV-2.

27. A nucleic acid encoding the immunogen of claim 1.

28. An expression vector comprising the nucleic acid of claim 27.

29. A host cell comprising the expression vector of claim 28.

30. A nucleic acid encoding the immunogen of claim 6.

31. An expression vector comprising the nucleic acid of claim 30.

32. A host cell comprising the expression vector of claim 31.

33. A vaccine comprising the immunogen of claim 1.

34. A vaccine comprising the immunogen of claim 6.

35. A method of treating COVID-19 in an individual, the method comprising administering to the individual a therapeutically effective amount of the vaccine of claim 33.

36. The method of claim 35, wherein the method further comprises administering to the individual at least one additional therapeutic agent for treating infection by SARS-CoV-2.

37. The method of claim 35, wherein the vaccine is administered to the individual before the individual manifests a symptom or clinical sign of infection by SARS-CoV-2.

38. The method of claim 35, wherein the vaccine is administered to the individual after the individual manifests a symptom or clinical sign of infection by SARS-CoV-2.

39. A method of treating COVID-19 in an individual, the method comprising administering to the individual a therapeutically effective amount of the vaccine of claim 34.

40. The method of claim 39, wherein the method further comprises administering to the individual at least one additional therapeutic agent for treating infection by SARS-CoV-2.

41. The method of claim 39, wherein the vaccine is administered to the individual before the individual manifests a symptom or clinical sign of infection by SARS-CoV-2.

42. The method of claim 39, wherein the vaccine is administered to the individual after the individual manifests a symptom or clinical sign of infection by SARS-CoV-2.


SEQUENCE LISTING (14 sequences)

SEQ ID NO: 1 - protein, 20 aa, synthetic construct
LQPELDSFKEELDKYFKNHT

SEQ ID NO: 2 - protein, 166 aa, synthetic construct
MALQPELDSFKEELDKYFKNHTSTGVGSASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYENDENDTPG

SEQ ID NO: 3 - protein, 468 aa, synthetic construct
METALALYSTHRILEVALLEUSERVALGLYGLUGLYTHRARGTHRLEUTHRGLUILEGLNSERTHRALAASPARGGLNILEPHEGLUGLULYSVALGLYPROLEUVALGLYARGLEUARGLEUTHRALASERLEUARGGLNASNGLYALALYSTHRALATYRARGVALASNLEULYSLEUASPGLNALAASPVALVALASPCYSSERTHRSERVALCYSGLYGLULEUPROLYSVALARGTYRTHRGLNVALTRPSERHISASPVALTHRILEVALALAASNSERTHRGLUALASERARGLYSSERLEUTYRASPLEUTHRLYSSERLEUVALALATHRSERGLNVALGLUASPLEUVALVALASNLEUVALPROLEUGLYARGGLYGLYPROSERGLUSERGLYALALEUGLNPROGLULEUASPSERPHELYSGLUGLULEUASPLYSTYRPHELYSASNHISTHR

SEQ ID NO: 4 - protein, 489 aa, synthetic construct
ATGGCTCTGCAGCCGGAACTGGACTCTTTCAAAGAAGAACTGGACAAATACTTCAAAAACCACACCTCAACCGGAGTTGGAAGCGCTTCTAACTTTACTCAGTTCGTTCTCGTCGACAATGGCGGAACTGGCGACGTGACTGTCGCCCCAAGCAACTTCGCTAACGGGGTCGCTGAATGGATCAGCTCTAACTCGCGTTCACAGGCTTACAAAGTAACCTGTAGCGTTCGTCAGAGCTCTGCGCAGAATCGCAAATACACCATCAAAGTCGAGGTGCCTAAAGTGGCAACCCAGACTGTTGGTGGTGTAGAGCTTCCTGTAGCCGCATGGCGTTCGTACTTAAATATGGAACTAACCATTCCAATTTTCGCTACGAATTCCGACTGCGAGCTTATTGTTAAGGCAATGCAAGGTCTCCTAAAAGATGGAAACCCGATTCCCTCAGCAATCGCAGCAAACTCCGGCATCTACTAATAGACGCCGGGTTAA

SEQ ID NO: 5 - protein, 471 aa, synthetic construct
ATGGCCAAAACCATCGTTCTTTCGGTCGGCGAGGGTACCCGCACTCTGACTGAGATCCAGTCCACCGCAGACCGTCAGATCTTCGAAGAGAAGGTCGGGCCTCTGGTGGGTCGGCTGCGCCTCACGGCTTCGCTCCGTCAAAACGGAGCCAAGACCGCGTATCGAGTCAACCTAAAACTGGATCAGGCGGACGTCGTTGATTGCTCCACCAGCGTCTGCGGCGAGCTTCCGAAAGTGCGCTACACTCAGGTATGGTCGCACGACGTGACAATCGTTGCGAATAGCACCGAGGCCTCGCGCAAATCGTTGTACGATTTGACCAAGTCCCTCGTCGCGACCTCGCAGGTCGAAGATCTTGTCGTCAACCTTGTGCCGCTGGGCCGTGGTGGTCCGTCTGAATCTGGTGCTCTGCAGCCGGAACTGGACTCTTTCAAAGAAGAACTGGACAAATACTTCAAAAACCACACCTAA

SEQ ID NO: 6 - protein, 17 aa, synthetic construct
KQRRPQGLPNNTASWFT

SEQ ID NO: 7 - protein, 10 aa, synthetic construct
LPKGFYAEGS

SEQ ID NO: 8 - protein, 12 aa, synthetic construct
RIRGGDGKMKDL

SEQ ID NO: 9 - protein, 13 aa, synthetic construct
KMSGKGQQQQGQT

SEQ ID NO: 10 - protein, 12 aa, synthetic construct
KQRRPQGLPNNT

SEQ ID NO: 11 - protein, 12 aa, synthetic construct
RRPQGLPNNTAS

SEQ ID NO: 12 - protein, 11 aa, synthetic construct
EDLKFPRGQGV

SEQ ID NO: 13 - protein, 10 aa, synthetic construct
PQGTTLPKGF

SEQ ID NO: 14 - protein, 11 aa, synthetic construct
ADLDDFSKQLQ